(19) United States
(12) Patent Application Publication
(10) Pub. No.: US 2024/0310334 A1
(43) Pub. Date: Sep. 19, 2024

(54) EXTRACTING VITAMINS B1 AND C FROM COMMIPHORA MYRRHA

(71) Applicant: KING FAISAL UNIVERSITY, AL-AHSA (SA)

(72) Inventors: AMEL YOUSIF IBRAHIM, AL-AHSA (SA); IMENE BAYACH, AL-AHSA (SA)

(21) Appl. No.: 18/122,556

(22) Filed: Mar. 16, 2023

(51) Int. Cl. G01N 30/06; B01D 11/02; C07D 307/62

(52) U.S. Cl. CPC G01N 30/06; B01D 11/0288; C07D 307/62; G01N 2030/062

(57) ABSTRACT

A method of extracting at least one vitamin from Commiphora myrrha selected from vitamin B1 (thiamin) and vitamin C (ascorbic acid) includes contacting a dried Commiphora myrrha resin with an extracting solvent to obtain an extract including the at least one vitamin. A vitamin selected from Vitamin B1 and Vitamin C from the extract can be isolated from the extract using high liquid performance chromatography (HPLC).

### BACKGROUND

### 1. Field

The disclosure of the present patent application relates to a method for extracting vitamins from Commiphora myrrha and, particularly, to a method for extracting Vitamin B1 and Vitamin C from Commiphora myrrha.

### 2. Description of the Related Art

Myrrh is an aromatic gum or resin made from the dried sap of trees and shrubs belonging to the Commiphora genus (Commiphora myrrha), of the Burseraceae family.

Recent studies have shown that myrrh is made of from 3% to 8% essential oils, from 25% to 40% of resins soluble in alcohol and from 30% to 60% of gums soluble in water. Some of the commonly known chemical components of the essential oils comprise monoterpenes, sesquiterpenes and small aromatic molecular compounds. Some of the commonly known chemical compounds of the resins comprise diterpenoids, triterpenoids, steroids and lignans.

Vitamin C, also known as L-ascorbic acid, is a water-soluble vitamin. Vitamin C in food is absorbed by the upper part of the human small intestine. Once absorbed, it is distributed to all water-soluble structures in the body. In normal adults, there is about 1500 mg of vitamin C in the vitamin C metabolic activity pool, and the highest storage peak is 3000 mg of vitamin C. In addition, the role of vitamin C is very large, as it can promote the biosynthesis of collagen, facilitate the faster healing of tissue wounds, promote the metabolism of tyrosine and tryptophan in amino acids, prolong the life of the body, and improve fat and lipids.

Vitamin B1 is generally in a white powder form that is easily soluble in water and easily decomposed in the presence of alkali. Vitamin B1 can be taken as a supplement to increase appetite and maintain normal nerve activity for example.

Both vitamin B1 and vitamin C, can possess antioxidant properties. Antioxidants play a significant role in scavenging free radicals and neutralizing their damaging effects, to thereby prevent disease.

Accordingly, a method of extracting Vitamin B1 and Vitamin C from Commiphora myrrha is desired.

### SUMMARY

The present subject matter relates to a method of extracting at least one vitamin from Commiphora myrrha. The at least one vitamin can be selected from vitamin B1 (thiamin) and vitamin C (ascorbic acid). In an embodiment, a sample of Commiphora myrrha can be dried, reduced to a powder, and contacted with a suitable extracting solvent to obtain an extract including the at least one vitamin. In an embodiment, the method further comprises isolating a vitamin selected from the group consisting of Vitamin B1, Vitamin C, and combinations thereof from the extract. In a specific embodiment, the vitamin is isolated using high liquid performance chromatography (HPLC). In other embodiments, further vitamins can be obtained from the Commiphora myrrha.

In an embodiment, a method of extracting at least one vitamin from Commiphora myrrha includes using an extracting solvent selected from an organic solvent and an alcohol solvent. In an embodiment, the organic solvent is selected from toluene and hexane. In an embodiment the alcohol solvent is selected from ethanol and methanol. In an embodiment, a Soxhlet extractor is used for extracting the at least one vitamin.

In one embodiment, the at least one vitamin may be readily added to food substances and medicinal agents without rendering the latter distasteful. In addition, isolating the at least one vitamin as described herein facilitates accurate control of the dosage of each vitamin to be administered.

These and other features of the present subject matter will become readily apparent upon further review of the following specification.

### BRIEF DESCRIPTION OF THE DRAWINGS

FIG. 1 is a chromatogram of standards solutions for Vitamins B1 and C.

FIG. 2 is a chromatogram of a myrrha resin sample in ethanol

FIG. 3 is a chromatogram of myrrha resin sample with methanol.

FIG. 4 is a chromatogram of a myrrha resin sample with toluene.

FIG. 5 is a chromatogram of a myrrha resin sample with hexane.

### DETAILED DESCRIPTION OF THE PREFERRED EMBODIMENTS

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

It will be understood by those skilled in the art with respect to any chemical group containing one or more substituents that such groups are not intended to introduce any substitution or substitution patterns that are sterically impractical and/or physically non-feasible.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The present subject matter relates to a method of extracting at least one vitamin from Commiphora myrrha. In an embodiment, the method can include collecting a sample of Commiphora myrrha resin, drying the resin, reducing the dried resin to a fine powder, and using an extraction method to obtain the at least one vitamin. The extraction method can include contacting the powdered resin with an extracting solvent selected from an organic solvent and an alcohol solvent. According to an embodiment, the at least one vitamin is selected from vitamin B1 (thiamin) and vitamin C (ascorbic acid). In an embodiment, the method of extracting at least one vitamin from Commiphora myrrha further includes isolating a vitamin selected from Vitamin B1 and Vitamin C from the extract.

In an embodiment, a method of extracting at least one vitamin from Commiphora myrrha includes contacting a sample of Commiphora myrrha resin with a suitable extracting solvent to produce an extract and isolating a vitamin selected from Vitamin B1 and Vitamin C from the extract. In a specific embodiment, the vitamin is isolated using high liquid performance chromatography (HPLC). In an embodiment, the HPLC retention time (RT) or amount of time for Vitamin B1 to pass through the chromatography column can be about 2.5 minutes to about 3 minutes. In an embodiment, the HPLC retention time (RT) for Vitamin C to pass through the chromatography column can be about 1.5 minutes to about 2 minutes. In an embodiment, the sample of Commiphora myrrha resin is a powdered resin.

In an embodiment, a sample of Commiphora myrrha resin can be dried and reduced to a powder to provide a powdered resin. The powdered resin can be contacted with a suitable extracting solvent to extract the at least one vitamin. In an embodiment, the extracting solvent is selected from an organic solvent and an alcohol solvent. In an embodiment, the organic solvent is selected from toluene and hexane. In an embodiment the alcohol solvent is selected from ethanol and methanol.

In an embodiment, the powdered Commiphora myrrha is contacted with the extracting solvent using a Soxhlet extractor. In an embodiment, the powdered resin can be placed inside a thimble and the thimble can be loaded into the main chamber of the Soxhlet extractor. The extracting solvent can be loaded into a distillation flask that is then heated. The Soxhlet extractor is placed on top of the flask and a reflux condenser is placed on top of the extractor. The solvent is heated to reflux to begin the extraction/distillation cycle. The extraction/distillation cycle can be repeated more than once until the extract including the at least one vitamin is concentrated in the distillation flask. In an embodiment, the extraction/distillation cycle can be repeated four times.

Commiphora myrrha has not previously been used as a source of vitamins for human consumption. The extract or at least one vitamin isolated from the extract may be readily added to food substances and medicinal agents. In an embodiment, the extract or at least one vitamin can be added to food substances and/or medicinal agents without altering the taste of the food substances and medicinal agents. In an embodiment, the extract or at least one vitamin isolated from the extract can be taken as a supplement for therapeutic and/or nutritional benefits. Isolating the at least one vitamin as described herein facilitates accurate control of the dosage each vitamin to be administered.

### EXAMPLES

### Example 1

### Vitamin Extraction

Samples from myrrha resin were dried and ground to a fine powder form. Vitamin extraction was conducted by a solvent extraction technique using a Soxhlet extractor. The solvents used included ethanol, methanol, toluene and hexane.

About 1 g of the Myrrh resin powder was weighed accurately and placed into the thimble in the chamber of the Soxhlet extractor. 100 ml of a selected solvent was placed in a round bottom flask and dispensed in the Soxhlet extractor and four cycles of extraction/distillation were conducted. The final extract was filtered and placed into a polyethene container.

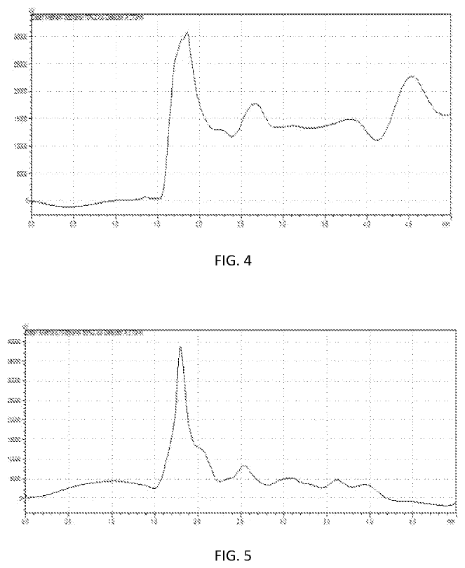

This method was conducted for each of the solvents, resulting in four extraction procedures. FIGS. 2-5 depict chromatograms of a myrrha resin sample in ethanol, methanol, toluene, and hexane, respectively.

### Example 2

### Sample Analysis

Eight samples of vitamins B1 and C were analyzed using High Liquid performance Chromatography, (Prominence-i (LC-2030, LIQUID CHROMATOGRAPH from SHIMADZU) with column C18 100 mm, UV detector and RF 20 detector.

Sample injectors (A) and (B) included: A 1.5 mL of sample; and B 1.5 mL of sample. Mobile phase containers (A) and (B) included: A: Water; and B: Methanol. A 440 bar LC-2030 pump, dual variable wavelength detector, heater/cooler oven and lab software solution consisting of LC-2030 Controller, LC-2030 Auto sampler and LC-2030 Auto purge were used.

The prepared samples as mentioned above were diluted in 10 ml sodium hydrogen phosphate buffer (NaHPO₄) (pH 3.5) and filtered over a 0.4 μm pore size syringe filter.

The mobile phase was prepared and consisted of 95%-NaHPO₄ (0.025M) pH 4.5 and 5% Methanol (HPLC grade quality).

10 μL of the standard and each sample was injected. The samples were separated at 30° C. at a flow rate of 1 mL/min with a linear gradient at wavelength 270 nm.

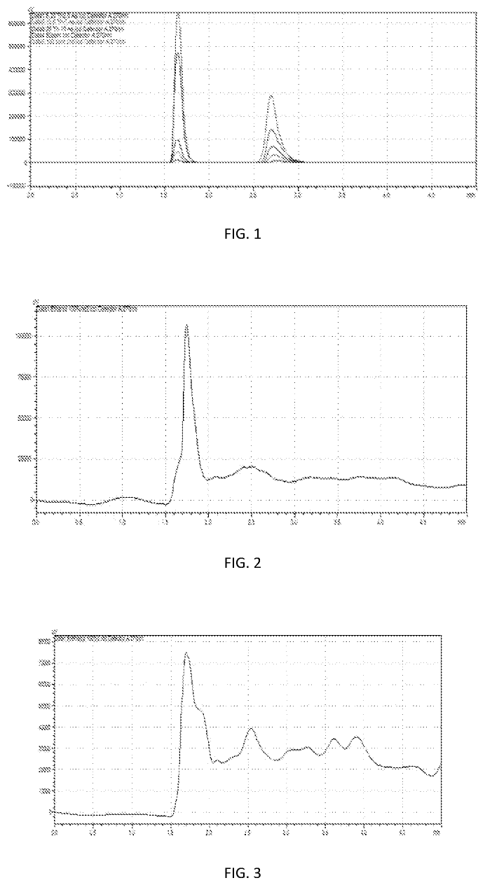

Stock standard solutions of Ascorbic Acid (Vitamin C) and Thiamine (Vitamin B1) (1000 μg/mL) were prepared by dissolving an appropriate amount directly in water and stored by keeping them protected from light and air at room temperature for a week. Working standard solutions were prepared daily. FIG. 1 is a chromatogram of standards solutions for Vitamins B1 and C.

The following Tables 1 and 2 provide summary statistics of Vitamin B1 and Vitamin C, respectively, obtained from Myrrh resin using different solvents.

TABLE 1
Summary Statistics of Vitamin B1 in
Myrrh resin using different solvents

| Solvent | Mean (ppm) | SD | LOD (ppm) | LOQ (ppm) |
|---|---|---|---|---|
| Ethanol | 11.19 | 2.87 | 2.23 | 6.76 |
| Methanol | 15.94 | 0.88 | | |
| Toluene | 5.76 | 0.96 | | |
| Hexane | 8.67 | 0.92 | | |

TABLE 2
Summary Statistics of Vitamin C in Myrrh
resin using different solvents

| Solvent | Mean (ppm) | SD | LOD (ppm) | LOQ (ppm) |
|---|---|---|---|---|
| Ethanol | 15.53 | 1.00 | 0.35 | 1.05 |
| Methanol | 9.89 | 2.03 | | |
| Toluene | 5.08 | 1.49 | | |
| Hexane | 2.39 | 0.55 | | |

It is to be understood that the present methods are not limited to the specific embodiments or examples described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1: A method of extracting at least one vitamin from Commiphora myrrha, comprising:
providing a dried resin of Commiphora myrrha;
reducing the dried resin to a powdered resin;
using an extraction method to obtain an extract from the powdered resin, the extract including at least one vitamin selected from the group consisting of Vitamin B1, Vitamin C, and combinations thereof; and
isolating the at least one vitamin from the extract.

2: The method of claim 1, further comprising:
contacting the powdered resin with an extracting solvent to obtain the extract.

3: The method of claim 2, wherein the extracting solvent is selected from the group consisting of an organic solvent and an alcohol solvent.

4: The method of claim 3, wherein the organic solvent is selected from the group consisting of toluene and hexane.

5: The method of claim 3, wherein the alcohol solvent is selected from the group consisting of ethanol and methanol.

6: The method of claim 2, wherein the powdered resin is contacted with the extracting solvent using a Soxhlet extractor.

7: The method of claim 6, wherein a cycle of extraction and distillation using the Soxhlet extractor is repeated four times.

8. (canceled)

9: The method of claim 1, wherein the at least one vitamin is isolated using high liquid performance chromatography (HPLC).

10: The method of claim 9, wherein the at least one vitamin is Vitamin B1 and HPLC retention time is about 2 minutes to about 3 minutes.

11: The method of claim 9, wherein the at least one vitamin is Vitamin C and HPLC retention time is about 1.5 minutes to about 2 minutes.

12-20. (canceled)
